(19) United States
(12) Patent Application Publication
(10) Pub. No.: US 2024/0139522 A1
(43) Pub. Date: May 2, 2024

(54) SELECTIVE ULTRA-LOW FREQUENCY STIMULATION THERAPY

(71) Applicant: Medtronic, Inc., Minneapolis, MN (US)

(72) Inventors: Leonid M. Litvak, Bet Shemesh (IL); Jeffery Michael Kramer, St. Louis Park, MN (US); Jerel Keith Mueller, St. Paul, MN (US)

(21) Appl. No.: 18/547,625

(22) PCT Filed: Feb. 23, 2022
(86) PCT No.: PCT/US2022/017527

§ 371 Date: Aug. 23, 2023

Related U.S. Application Data
(60) Provisional application No. 63/153,263, filed on Feb. 24, 2021.

(51) Int. Cl. A61N 1/36

(52) U.S. Cl. CPC A61N 1/36178; A61N 1/36171

(57) ABSTRACT

In some examples, the disclosure relates to system, devices, and techniques for delivering electrical stimulation therapy to treat patient disorders. In some example, the disclosure is directed to a method including controlling, using processing circuitry, the delivery of an electrical stimulation therapy to a patient via a medical device, wherein the electrical stimulation therapy includes a plurality of bi-phasic pulses, each pulse of the di¬phasic pulses including a first phase followed by a second phase, and wherein the plurality 7 of bi-phasic pulses are configured to substantially block transmission of neural activity′ along nerve fibers.

This application claims the benefit of U.S. Provisional Patent Application No. 63/153,263, filed Feb. 24, 2021, the entire content of which is incorporated by reference herein.

### TECHNICAL FIELD

The disclosure relates to medical devices and, more particularly, to programmable medical devices that deliver electrical stimulation therapy to a patient.

### BACKGROUND

A variety of therapies, such as neurostimulation or therapeutic agents, e.g., drugs, may be delivered to a patient to treat chronic or episodic pain. Neurostimulation is typically delivered by an implantable medical device (IMD). An IMD delivers neurostimulation therapy via electrodes, which are coupled to the IMD by one or more leads, or carried by the IMD housing in the case of a leadless stimulator. The number and positions of the leads and electrodes is largely dependent on the type or cause of the pain, and the type of neurostimulation delivered to treat the pain. In general, an IMD may deliver neurostimulation therapy in the form of electrical stimulation signals such as pulses and continuous waveforms.

### SUMMARY

In general, the disclosure is directed to systems, devices and techniques for delivering electrical stimulation therapy to a patient.

In one example, the disclosure is directed to a method comprising: controlling, using processing circuitry, the delivery of an electrical stimulation therapy to a patient via a medical device, wherein the electrical stimulation therapy includes a plurality of bi-phasic pulses, each pulse of the bi-phasic pulses including a first phase followed by a second phase, and wherein the plurality of bi-phasic pulses are configured to substantially block transmission of neural activity along nerve fibers of the patient.

In another example, the disclosure is directed to a medical device system comprising: a therapy module configured to deliver electrical stimulation therapy to a patient; and processing circuitry configured to control the therapy module to deliver the electrical stimulation to the patient such that the electrical stimulation therapy includes a plurality of bi-phasic pulses, each pulse of the bi-phasic pulses including a first phase followed by a second phase, and wherein the plurality of bi-phasic pulses are configured to substantially block transmission of neural activity along nerve fibers of the patient.

The details of one or more examples of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a conceptual diagram illustrating an example implantable stimulation system.

FIG. 2 is a functional block diagram illustrating various components of an example implantable electrical stimulator.

FIG. 3 is a functional block diagram illustrating various components of an example external programmer for an implantable medical device.

FIGS. 4A, 4B, 5A, 5B and 6A-6D are example timing diagrams illustrating various example waveforms in accordance with examples of the disclosure.

FIG. 7 is a flow diagram illustrating an example technique for controlling the delivery of stimulation to a patient in accordance with examples of the disclosure.

### DETAILED DESCRIPTION

In general, the disclosure is directed to systems, devices and techniques for delivering electrical stimulation therapy to a patient. In some examples, the therapy may include the delivery of ultra-low frequency (also referred to as “ULF”) waveforms to a patient for neural modulation. In some examples, the ULF waveforms are designed for application to complex neural structures. Examples may include peripheral nerves (which contain a mixture of fiber types such as A, B and C fibers) of the patient, dorsal root ganglia and/or, and the spinal cord of the patient. The ULF waveforms may include a series of bi-phasic waveforms (referred to in some examples as “bi-phasic pulses”) configured to block neural activity from being conducted along the fibers (e.g., from one end of the fiber to the other). In addition, ULF waveform will not in itself excite neural elements in a manner that causes them to generate neural activity or spiking. The individual bi-phasic waveforms may be substantially charge balanced and have relatively long pulse width (e.g., greater than 0.25 seconds) with one phase (e.g., cathodic phase) being longer than the other phase (e.g., anodic phase) but with lower amplitude than the other phase.

Because ULF waveform may be less effective in blocking potentials during the transitions, e.g., between the phases, in some examples, higher frequency stimulation (e.g., stimulation with bursts of pulses delivered at a frequency greater than 1 kHz) may be delivered during a transition between the phases of the ULF stimulation, e.g., during the time at which the ULF may not successfully block the neural activity along the fibers as a result of the transmission. In some examples, the amplitude may be ramped up and ramped down for the respective phases of the ULF pulses, e.g., to prevent onset and offset activation of neural activity in the fibers that may otherwise result from the rapid increase or decrease in the stimulation amplitude. In some examples, one or more “gaps” (also referred to as “breaks”) in stimulation may be included during the cathodic and/or anodic phases of the bi-phasic ULF pulses. The gaps may by discrete periods during which the amplitude of the stimulation is reduced to zero or even reversed in polarity to some degree, e.g., to temporarily release a block of neural activity for A fibers but not release a block of neural activity for the C fibers. This may allow for the conduction of sensory information during the gaps. The gaps may end prior to the C fiber neural activity block being released (which may take longer to release than the A fiber neural activity block due to slower membrane time constant for C fibers relative to A fibers and increased carry over effects of the delivered stimulation).

In some examples, the respective phases of the bi-phasic ULF pulses may be configured to have a pulse width and amplitude that block the neural activity from being transmitted along the nerve fibers but with a pulse width and amplitude that does not result in undesirable chemical reactions that may cause degradation at the surface of the electrodes delivering the stimulation. Put another way, a chemical reaction may result when the total charge of one of the phases of a bi-phasic pulse reaches a threshold and that chemical reaction degrades a surface of electrode. Each respective phase of the bi-phasic pulse may have a relatively long width (the length of time the phase is delivered) and amplitude that blocks neural activity, but the phase does not have a total charge that is at or above such a threshold that chemical reaction degrades a surface of electrode. Thus, one goal of the ULF stimulation may be to deliver stimulation that blocks activity from traveling along the nerve fibers and that is substantially charge balanced between the phases without degrading electrode material as described herein, e.g., due to undesired chemical reactions. This can be achieved by applying super-high capacitance coatings onto electrode surface, including titanium nitride (TiN), iridium oxide (IrOx), conductive polymer PEDOT-based coating (e.g., the proprietary MPEDOT coating) or by laser texturing/restructuring electrode surface or by combination of laser texturing/restructuring followed by addition of conductive polymer PEDOT-based coating. Additionally, or alternatively, the electrical stimulation described herein may be configured to preferentially block smaller nerve fibers (e.g., C-fibers) while allowing information to pass through larger fibers (e.g., A-fibers).

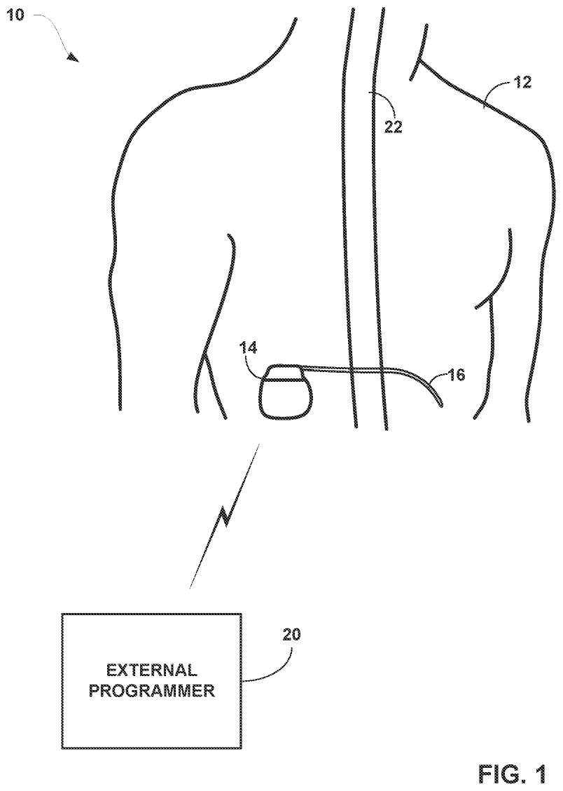

FIG. 1 is a schematic diagram illustrating an example implantable stimulation system 10 configured to delivery electrical stimulation to patient 12. Although the techniques described in this disclosure are generally applicable to a variety of medical devices including external and implantable medical devices (IMDs), application of such techniques to IMDs and, more particularly, implantable electrical stimulators such as neurostimulators will be described for purposes of illustration. More particularly, the disclosure will refer to an implantable neurostimulation system for purposes of illustration, but without limitation as to other types of medical devices.

As shown in FIG. 1, system 10 includes an IMD 14 and external programmer 20 shown in conjunction with a patient 12, who is ordinarily a human patient. In the example of FIG. 1, IMD 14 is an implantable electrical stimulator that delivers neurostimulation therapy to patient 12, e.g., for relief of chronic pain or other symptoms. In some examples, IMD 14 may deliver stimulation therapy to patient 12 to treat one or more sensory or motor disorders characterized at least in part by overactive nerve activity. Again, although FIG. 1 shows an IMD, other examples may include an external stimulator, e.g., with percutaneously implanted leads.

Electrical stimulation energy, which may be constant current or constant voltage based pulses, for example, is delivered from IMD 14 to one or more targeted locations within patient 12 via one or more electrodes (not shown) of implantable lead 16. The parameters for a program that controls delivery of stimulation energy by IMD 14 may include information identifying which electrodes have been selected for delivery of stimulation according to a stimulation program, the polarities of the selected electrodes, i.e., the electrode configuration for the program, and voltage or current amplitude, pulse rate, pulse shape, pulse width of stimulation delivered by the electrodes, and duty cycle. Delivery of stimulation pulses will be described for purposes of illustration. However, stimulation may be delivered in other forms, such as continuous waveforms.

In the example of FIG. 1, lead 16 may carry one or more electrodes that are placed adjacent to the target tissue. One or more electrodes may be disposed at a distal tip of lead 16 and/or at other positions at intermediate points along lead 16, for example. Electrodes of lead 16 transfer electrical stimulation generated by an electrical stimulation generator in IMD 14 to tissue of patient 12. The electrodes may be electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for therapy. Unipolar electrode configurations, in general, include one or more electrodes on one or more leads (e.g., one or more cathodes on the lead or leads) and one or more electrodes on a housing of IMD 14 (e.g., an anode on the housing). Bipolar and multipolar electrode configurations, in general, include multiple electrodes on one or more leads (e.g., one or more cathodes and one or more anodes on the lead or leads). In general, ring electrodes arranged at different axial positions at the distal ends of lead 16 will be described for purposes of illustration.

Lead 16 may be configured to deliver stimulation energy generated by IMD 14 to stimulate one or more peripheral nerves of patient 12, e.g., in the form of peripheral nerve stimulation (PNS). PNS may be used to treat patients suffering from intractable pain secondary to nerve damage isolated to a single nerve. PNS may include locating a group of electrodes in very close proximity to, e.g., in contact with, and approximately parallel to a major nerve in the subcutaneous tissue. PNS may also include placing a group of electrodes in very close proximity to a nerve that may be deeper in the limb, sometimes near to blood vessels. Placing electrodes in very close proximity to the nerve may ensure or increase the likelihood that only fibers within that nerve are activated at low amplitudes.

PNS electrodes may be located on percutaneous leads, but for stability and to prevent stimulation of other tissues proximate to the target peripheral nerve, PNS electrodes may be located within insulative material that wraps around a nerve, i.e., in so-called cuff electrodes, or on one surface of a flat paddle of insulative material placed under a nerve, i.e., forming a so-called paddle lead. In any case, the electrodes for PNS may be placed in close proximity to the nerve proximal from the source of damage or pain, e.g., closer to the spinal cord than the region of damage or pain. Upper extremity nerves that may be treated with PNS include the ulnar nerve, median nerve, radial nerve, tibial nerve, occipital nerve, and common peroneal nerve. When PNS is delivered to treat pain, one or more electrodes may be implanted proximate to or in contact with a specific peripheral nerve or branch that is responsible for the pain sensation.

IMD 14 may deliver electrical stimulation therapy to one or more nerve sites of patient 12 to treat or manage sensory and/or motor disorders. In some examples, IMD 14 may deliver therapy to treat one or more patient disorders characterized by pathological overactive afferent or efferent activity. Example sensory disorders that may be characterized by overactive afferent nerve activity may include chronic pelvic pain, interstitial cystitis, abacterial chronic prostatitis (Type IIB), neuralgias, and other chronic pain conditions. In such cases, the abnormal overactive afferent activity may cause pain, overwhelm central processing and inhibit associated neural activities through reflex pathways. Example motor disorders that may be characterized by overactive afferent nerve activity may include spasticity, tics, choreas, intractable hiccups and the like. IMD 14 may deliver electrical stimulation therapy to one or more nerve sites of patient 12 to block “normal” activity of a patient. For example, IMD 14 may deliver electrical stimulation therapy to one or more nerve sites of patient 12 to block nerve going to the liver for a diabetic patient to ensure that patient 12 does not produce excessive sugar.

In some examples, to treat such disorders, IMD 14 may deliver ultra-low frequency stimulation (e.g., PNS) to patient 12 via lead 16, e.g., alone or in combination with the periodic delivery of higher frequency stimulation to patient 12. IMD 14 may be configured to deliver the higher frequency stimulation to the same nerve site as the ultra-low frequency stimulation. The ultra-low frequency and/or higher frequency stimulation may be configured to substantially block nerve activity (e.g., block pathological nerve activity). While examples of the disclosure are primarily described with regard to PNS, examples are not limited as such. For example, IMD 14 may be configured to deliver electrical stimulation to one or more spinal cord nerve sites (including nerve root stimulation) in addition to or as an alternative to peripheral nerve sites. In some examples, the stimulation may take of the form of deep brain stimulation (DBS), peripheral nerve field stimulation (PNFS), subcutaneous electrical stimulation (SQS), autonomic nerve stimulation, spinal cord stimulation, transcutaneous electrical nerve stimulation (TENS) and/or organ stimulation.

Lead 16 within patient 12 may be directly or indirectly (e.g., via a lead extension) coupled to IMD 14. Alternatively, as mentioned above, lead 16 may be implanted and coupled to an external stimulator, e.g., through a percutaneous port. In some cases, an external stimulator is a trial or screening stimulation that is used on a temporary basis to evaluate potential efficacy to aid in consideration of chronic implantation for a patient. In additional examples, IMD 14 may be a leadless stimulator with one or more arrays of electrodes arranged on a housing of the stimulator rather than leads that extend from the housing.

IMD 14 delivers electrical stimulation therapy to patient 12 via selected combinations of electrodes carried by lead 16. The target tissue for the electrical stimulation therapy may be any tissue affected by electrical stimulation energy, which may be in the form of electrical stimulation pulses or waveforms. Again, while examples of the disclosure are primarily described with regard to PNS, target nerve sites may include nerve sites of the spinal cord 22, including dorsal column and dorsal root nerves. For example, in addition to or as an alternative to delivering stimulation to one or more peripheral nerves, nerve sites for electrical stimulation delivered via lead 18 may be part of spinal cord 22. In some examples, the target nerve sites for electrical stimulation delivered via lead 16 may be a dorsal root or other nerve roots that branch off spinal cord 22. Lead 16 may be introduced proximate spinal cord 22 via any suitable region, such as the thoracic, cervical or lumbar regions.

The deployment of electrodes via lead 16 is described for purposes of illustration, but arrays of electrodes may be deployed in different ways. For example, a housing associated with a leadless stimulator may carry arrays of electrodes, e.g., rows and/or columns (or other patterns). Such electrodes may be arranged as surface electrodes, ring electrodes, or protrusions. As a further alternative, electrode arrays may be formed by rows and/or columns of electrodes on one or more paddle leads. In some examples, electrode arrays may include electrode segments, which may be arranged at respective positions around a periphery of a lead, e.g., arranged in the form of one or more segmented rings around a circumference of a cylindrical lead.

The electrical stimulation delivered by IMD 14 may take the form of electrical stimulation pulses or continuous stimulation waveforms, and may be characterized by controlled voltage levels or controlled current levels, as well as pulse width and pulse rate (also referred to as pulse frequency). In the case of stimulation including envelopes or bursts including a plurality of pulses, the envelopes may be characterized by rate, and/or duration.

In some examples, IMD 14 generates and delivers stimulation therapy according to one or more programs. A program defines values for one or more parameters that define an aspect of the therapy delivered by IMD 14 according to that program. For example, a program that controls delivery of stimulation by IMD 14 in the form of pulses may define a voltage or current pulse amplitude, a pulse width, a pulse rate (i.e., frequency), for stimulation pulses delivered by IMD 14 according to that program. Moreover, therapy may be delivered according to multiple programs, wherein multiple programs are contained within each of a plurality of groups.

Each program group may support an alternative therapy selectable by patient 12, and IMD 14 may deliver therapy according to the multiple programs. IMD 14 may rotate through the multiple programs of the group when delivering stimulation such that numerous conditions of patient 12 are treated. As an illustration, in some cases, stimulation pulses formulated according to parameters defined by different programs may be delivered on a time-interleaved basis. For example, a group may include a program directed to leg pain, a program directed to lower back pain, and a program directed to abdomen pain. Alternatively, multiple programs may contribute to an overall therapeutic effect with respect to a particular type or location of pain. In this manner, IMD 14 may treat different symptoms substantially simultaneously or contribute to relief of the same symptom.

A user, such as a clinician or patient 12, may interact with a user interface of external programmer 20 to program IMD 14. Programming of IMD 14 may refer generally to the generation and transfer of commands, programs, or other information to control the operation of IMD 14. For example, external programmer 20 may transmit programs, parameter adjustments, program selections, group selections, or other information to control the operation of IMD 14, e.g., by wireless telemetry. Again, a program may be characterized by an electrode combination, electrode polarities, voltage or current amplitude, pulse width, pulse rate, pulse shape, envelope frequency, and/or envelope duration. A group may be characterized by multiple programs that are delivered simultaneously or on an interleaved or rotating basis.

In some cases, external programmer 20 may be characterized as a physician or clinician programmer if it is primarily intended for use by a physician or clinician. In other cases, external programmer 20 may be characterized as a patient programmer if it is primarily intended for use by a patient. A patient programmer is generally accessible to patient 12 and, in many cases, may be a portable device that may accompany the patient throughout the patient's daily routine. In general, a physician or clinician programmer may support selection and generation of programs by a clinician for use by stimulator 14, whereas a patient programmer may support adjustment and selection of such programs by a patient during ordinary use.

IMD 14 may be constructed with a biocompatible housing, such as titanium or stainless steel, or a polymeric material such as silicone or polyurethane, and surgically implanted at a site in patient 12 near the pelvis. IMD 14 may also be implanted in patient 12 at a location minimally noticeable to patient 12. Alternatively, TMD 14 may be external with percutaneously implanted lead(s). For spinal cord stimulation (SCS) or PNS, IMD 14 may be located in the lower abdomen, lower back, upper buttocks, or other location to secure IMD 14. Lead 16 may be tunneled from IMD 14 through tissue to reach a location adjacent to a target nerve site for stimulation delivery.

Implantable stimulation system 10 is not limited to that of one leads, but instead may include zero, two, three, four, five or more than five leads. For example, system 10 may include a second lead in addition to lead 16. In such a configuration, IMD 14 may deliver stimulation via combinations of electrodes carried by both leads, or a subset of the two leads. The electrode configuration may be multipolar (e.g., bipolar) or unipolar arrangements. The second lead may include a greater number of electrodes than lead 16 and be positioned on either side of lead 16. The number and configuration of all leads may be stored within external programmer 20 to allow programmer 20 to appropriately program stimulation therapy or assist in the programming of stimulation therapy.

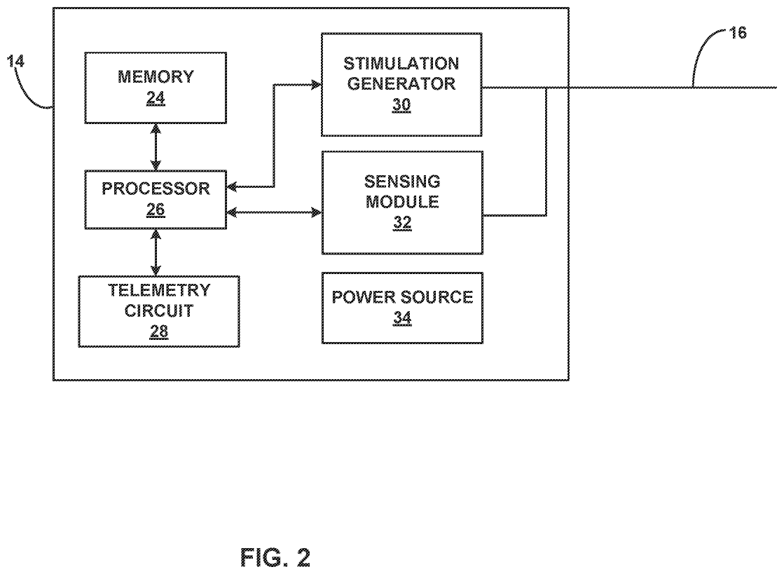

FIG. 2 is a functional block diagram illustrating various components of an IMD 14. In the example of FIG. 2, IMD 14 includes memory 24, processor 26, telemetry circuit 28, stimulation generator 30, sensing module 32, and power source 34. The stimulation generator 30 forms what may also be referred to as a therapy delivery module.

Memory 24 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Memory 24 may store instructions for execution by processor 26, stimulation therapy data, information regarding evoked signals sensed at one or more locations on the dorsal columns, and any other information regarding therapy or patient 12. Therapy information may be recorded for long-term storage and retrieval by a user, and the therapy information may include any data created by or stored in IMD 14. Memory 24 may include separate memories for storing instructions, sensed signal information, program histories, and any other data that may benefit from separate physical memory modules.

Memory 24 may be considered, in some examples, a non-transitory computer-readable storage medium comprising instructions that cause one or more processors, such as, e.g., processor 26, to implement one or more of the example techniques described in this disclosure. The term “non-transitory” may indicate that the storage medium is not embodied in a carrier wave or a propagated signal. However, the term “non-transitory” should not be interpreted to mean that memory 24 is non-movable. As one example, memory 24 may be removed from IMD 14, and moved to another device. In certain examples, a non-transitory storage medium may store data that can, over time, change (e.g., in RAM).

Processor 26, which may include processing circuitry, controls stimulation generator 30 to deliver electrical stimulation via electrode combinations formed by electrodes. For example, stimulation generator 30 may deliver electrical stimulation therapy via one or more electrodes of leads 16, e.g., as stimulation pulses or continuous waveforms. Components described as processors within IMD 14, external programmer 20 or any other device described in this disclosure may each comprise one or more processors, such as one or more microprocessors, digital signal processors (DSPs), application specific integrated circuits (ASICs), field programmable gate arrays (FPGAs), programmable logic circuitry, or the like, either alone or in any suitable combination. The functions attributed to processors described herein may be embodied as software, firmware, hardware, or any combination thereof.

Stimulation generator 30 may include stimulation generation circuitry to generate stimulation pulses or waveforms and switching circuitry to switch the stimulation across different electrode combinations, e.g., in response to control by processor 26. In particular, processor 26 may control the switching circuitry on a selective basis to cause stimulation generator 30 to deliver electrical stimulation to selected electrode combinations and to shift the electrical stimulation to different electrode combinations in a first direction or a second direction when the therapy must be delivered to a different location within patient 12. In other examples, stimulation generator 30 may include multiple current sources to selectively drive individual electrodes and deliver stimulation via more than one electrode combination at one time. In this case, stimulation generator 30 may decrease current to the first electrode combination and simultaneously increase current to the second electrode combination to shift the stimulation therapy.

An electrode configuration, e.g., electrode combination and associated electrode polarities, may be represented by data stored in a memory location, e.g., in memory 24, of IMD 14. Processor 26 may access the memory location to determine the electrode combination and control stimulation generator 30 to deliver electrical stimulation via the indicated electrode combination. To adjust electrode combinations, as well as amplitudes, pulse rates (frequency), or pulse widths, processor 26 may command stimulation generator 30 to make the appropriate changes to therapy according to instructions within memory 24 and rewrite the memory location to indicate the changed therapy. In other examples, rather than rewriting a single memory location, processor 26 may make use of two or more memory locations. When activating stimulation, processor 26 may access not only the memory location specifying the electrode combination but also other memory locations specifying various stimulation parameters such as voltage or current amplitude, pulse width and pulse rate (frequency). Stimulation generator 30, e.g., under control of processor 26, then makes use of the electrode combination and parameters in formulating and delivering the electrical stimulation to patient 12.

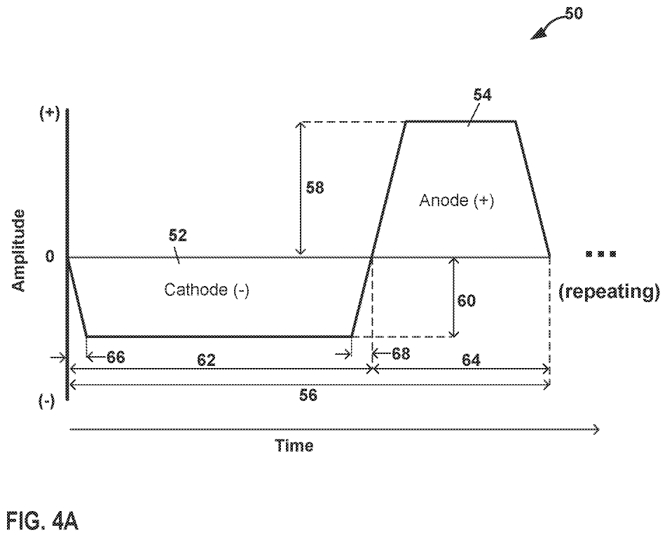

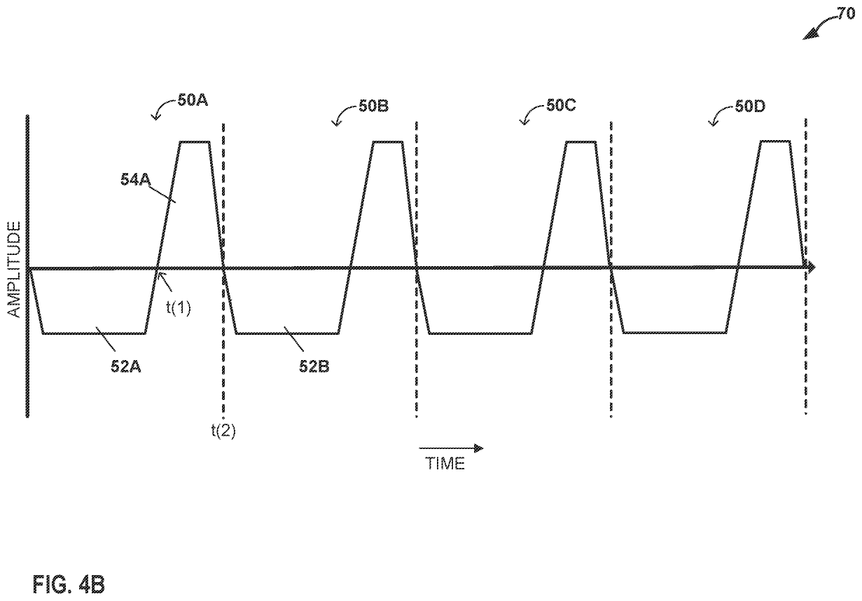

As described above, in some examples, IMD 14 may deliver stimulation including bi-phasic (asymmetric bi-phasic) pulses at an ultra-low frequency (e.g., from about 0.01 Hz to about 10 Hz, such as about 4 Hz or lower, such as 2 Hz or lower or 1 Hz or lower). Hence, ultra-low frequency waveforms may have a pulse frequency of greater than zero, and less than or equal to 4 Hz, less than or equal to 2 Hz, or less than or equal to 1 Hz. The ULF stimulation may be delivered alone, i.e., without non-ULF stimulation, or in combination with non-ULF stimulation such as higher frequency stimulation to treat one or more patient disorders. The higher frequency stimulation may include discrete periods of time in which a plurality of pulses are delivered at a frequency of at least about 1 kHz in combination with the ULF stimulation pulses. IMD 14 may deliver stimulation in accordance with the examples described with regard to FIGS. 4A-7. The electrical stimulation delivered by IMD 14 to patient 12 may be configured to block nerve activity of patient 12, e.g., at or near the target site of the stimulation. The electrical stimulation may be delivered to at least partially (e.g., substantially fully) block nerve activity of patient 12 at or near the target site of the stimulation. In the case of partial block, while the response in each individual nerve fiber to the stimulation may be binary (blocked/unblocked), the stimulation may not block every nerve fiber (e.g., of a bundle or group of nerve fibers) so that the nerve activity of a bundle/group of fibers is only partially blocked.

Processor 26 accesses stimulation parameters in memory 24, e.g., as programs and groups of programs. Upon selection of a particular program group, processor 26 may control stimulation generator 30 to generate and deliver stimulation according to the programs in the groups, e.g., simultaneously or on a time-interleaved basis. A group may include a single program or multiple programs. As mentioned previously, each program may specify a set of stimulation parameters, such as amplitude, pulse width and pulse rate. In addition, each program may specify a particular electrode combination for delivery of stimulation. Again, the electrode combination may specify particular electrodes in a single array or multiple arrays, e.g., on a single lead or among multiple leads. Processor 26 also may control telemetry circuit 28 to send and receive information to and from external programmer 20.

Sensing module 32 may be configured to monitor, with sensing circuitry, one or more signals from one or more electrodes on lead 16 in order to monitor electrical activity at one more locations in patient 12, e.g., via electrogram (EGM) signals. For example, sensing module 32 may be configured to, using sensing circuitry, monitor one or more electrical signals from electrode(s) on lead 16 at nerve site locations. Such electrical signals may be intrinsic or evoked by delivery of stimulation by IMD 14. Signals sensed via a particular electrode may be made with reference to another electrode on a lead or an electrode on the housing of IMD 16. Sensing module 32 may also include a switch module to select which of the available electrodes, or which pairs or combinations of electrodes, are used to sense intrinsic activity or activity evoked, e.g., by PNS.

Signals produced by the sense amplifiers may be converted from analog signals to digital signals by analog-to-digital converters (ADCs) provided by sensing module 32. The digital signals may be stored in memory for analysis on-board the IMD 14 or remote analysis by a programmer 20 or other device. Sensing module 32 may include a digital signal processor (DSP) that implements any of a variety of digital signal processing features such as digital amplifiers, digital filters, and the like.

IMD 14 wirelessly communicates with external programmer 20, e.g., a patient programmer or a clinician programmer, or another device by radio frequency (RF) communication or proximal inductive interaction of IMD 14 with external programmer 20. Telemetry circuit 28 may send information to and receive information from external programmer 20 on a continuous basis, at periodic intervals, at non-periodic intervals, or upon request from the stimulator or programmer. To support RF communication, telemetry circuit 28 may include appropriate electronic components, such as one or more antennas, amplifiers, filters, mixers, encoders, decoders, and the like.

Power source 34 delivers operating power to the components of IMD 14. Power source 34 may include a small rechargeable or non-rechargeable battery and a power generation circuit to produce the operating power. Recharging may be accomplished through proximal inductive interaction between an external charger and an inductive charging coil within IMD 14. In some examples, power requirements may be small enough to allow IMD 14 to utilize patient motion and implement a kinetic energy-scavenging device to trickle charge a rechargeable battery. In other examples, traditional batteries may be used for a limited period of time. As a further alternative, an external inductive power supply could transcutaneously power IMD 14 when needed or desired.

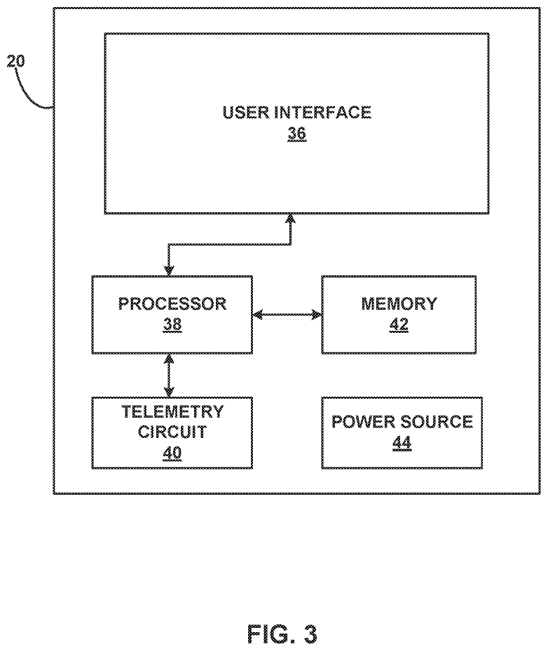

FIG. 3 is a functional block diagram illustrating various components of an external programmer 20 for IMD 14. Programmer 20 may be a handheld computing device, a workstation or another dedicated or multifunction computing device. For example, programmer 20 may be a general purpose computing device (e.g., a personal computer, personal digital assistant (PDA), cell phone, and so forth) or may be a computing device dedicated to programming the IMD. As shown in FIG. 3, external programmer 20 includes user interface 36, processor 38, telemetry circuit 40, memory 42, and power source 44. External programmer 20 may be embodied as a patient programmer or clinician programmer.

Processor 38 processes instructions by memory 42 and may store user input received through user interface 36 into the memory when appropriate for the current therapy. In addition, processor 38 provides and supports any of the functionality described herein with respect to each example of user interface 36. Processor 38 may comprise any one or more of a microprocessor, DSP, ASIC, FPGA, or other digital logic circuitry, and the functions attributed to programmer 38 may be embodied as software, firmware, hardware or any combination thereof.

Memory 42 may include any one or more of a RAM, ROM, EEPROM, flash memory or the like. Memory 42 may include instructions for operating user interface 36, telemetry module 40 and managing power source 44. Memory 42 may store program instructions that, when executed by processor 38, cause processor 38 and programmer 20 to provide the functionality ascribed to them herein. Memory 42 also includes instructions for generating and delivering programming commands to IMD 14. Memory 42 may also include a removable memory portion that may be used to provide memory updates or increases in memory capacities. A removable memory may also allow patient data to be easily transferred to another computing device, or to be removed before programmer 20 is used to program therapy for another patient.

Memory 42 may be considered, in some examples, a non-transitory computer-readable storage medium comprising instructions that cause one or more processors, such as, e.g., processor 38 and/or processor 26, to implement one or more of the example techniques described in this disclosure. The term “non-transitory” may indicate that the storage medium is not embodied in a carrier wave or a propagated signal. However, the term “non-transitory” should not be interpreted to mean that memory 42 is non-movable. As one example, memory 42 may be removed from IMD programmer 20, and moved to another device. In certain examples, a non-transitory storage medium may store data that can, over time, change (e.g., in RAM).

A clinician, patient 12, or another user (e.g., a patient caretaker) interacts with user interface 36 in order to manually change the stimulation parameter values of a program, change programs within a group, or otherwise communicate with IMD 14. User interface 36 may include a screen and one or more mechanisms, such as, buttons, as in the example of a patient programmer, that allow external programmer 20 to receive input from a user. Alternatively, user interface 36 may additionally or only utilize a touch screen display, as in the example of a clinician programmer. The screen may be a liquid crystal display (LCD), dot matrix display, organic light-emitting diode (OLED) display, touch screen, or any other device capable of delivering and/or accepting information.

Processor 38 controls user interface 36, retrieves data from memory 42 and stores data within memory 42. Processor 38 also controls the transmission of data through telemetry circuit 40 to IMDs 14 or 26. Memory 42 includes operation instructions for processor 38 and data related to delivery of therapy to patient 12.

Telemetry circuit 40 allows the transfer of data to and from IMD 14. Telemetry circuit 40 may communicate automatically with IMD 14 at a scheduled time or when the telemetry circuit detects the proximity of the stimulator. Alternatively, telemetry circuit 40 may communicate with IMD 14 when signaled by a user through user interface 36. To support RF communication, telemetry circuit 40 may include appropriate electronic components, such as amplifiers, filters, mixers, encoders, decoders, and the like. Power source 44 may be a rechargeable battery, such as a lithium ion or nickel metal hydride battery. Other rechargeable or conventional batteries may also be used. In some cases, external programmer 20 may be used when coupled to an alternating current (AC) outlet, i.e., AC line power, either directly or via an AC/DC adapter. Although not shown in FIG. 3, in some examples, external programmer 20 may include a charger module capable of recharging a power source, such as a rechargeable battery that may be included in power source 34 of IMD 14. Hence, in some cases, the programmer may be integrated with recharging components to form a combined programmer/recharger unit.

FIG. 4A is an example timing diagram showing the delivery of an example, single pulse 50 (bi-phasic pulse) used for the delivery of ULF stimulation in accordance with examples of the disclosure. Pulse 50 is a bi-phasic pulse with cathodic phase 52 and anodic phase 54. Examples of the disclosure may include the continuous delivery of such a pulse in a repeating fashion (e.g., without or without a time delay between the end of the pulse 50 shown in FIG. 4 and the next subsequent pulse having the same waveform). Since the bi-phasic pulse 50 may have a relatively long pulse width 56 (e.g., a pulse width defined by the combined length of both phases 52 and 54 of at least about 0.25 seconds such as at least about 0.5 seconds or at least about 1 second and/or less than or equal to about 20 seconds or about 100 seconds), the repeated and continuous delivery of the bi-phasic pulse 50, e.g., as a series of such bi-phasic pulses, may be at a relatively low frequency (e.g., from about 0.01 Hz to about 10 Hz, such as about 4 Hz or lower, such as 2 Hz or 1 Hz or lower). The example waveform in FIG. 4A may be referred to as “Waveform 1.” FIG. 4B is an example timing diagram showing the repeated and continuous delivery of bi-phasic pulse 50, i.e., in a series of pulses 50A, 50B, etc., over a period of time.

Waveform 1 may be referred to as an asymmetric waveform. The anodic phase 54 of the pulse waveform has a higher amplitude (current or voltage amplitude) than the cathode phase 52 of the pulse waveform. In some examples, the rationale for such a waveform is that electrical stimulation delivered via an anode requires a higher level to reach a block (e.g., a block of nerve propagation or activation) compared to electrical stimulation delivered via a cathode. Unlike other waveforms in which the cathodic and anodic phases have equal amplitude or the cathodic phase has a greater amplitude, the waveform of pulse 50 in FIG. 4A, in which the cathodic phase has a lesser amplitude than the anodic phase, may obtain a more consistent block. To maintain or approximate charge balance, the cathodic phase 52 is made longer in duration 62 (i.e., width) than the duration 64 of the anodic phase 54. In some examples, cathodic phase 52 has an amplitude of about 0.1V to about 10V. In some examples, the cathodic phase 52 has an amplitude of about 1 milliamp (mA) to about 10 mA

In some examples, as shown in FIG. 4A, the waveform of pulse 50 may include a gradual ramp in amplitude (e.g., as opposed to a substantially instantaneous increase or decrease in amplitude) at the onset or offset of cathodic phase 52, anodic phase 54, and/or overall pulse waveform. The gradual ramp in amplitude may be applied to avoid exciting the neurons due to the rapid increase or decrease in amplitude of the stimulation. In some examples, a ramp up in amplitude is to avoid onset activation of neurons in response to the drastic change in amplitude. In some examples, a ramp down in amplitude is to avoid anodic/cathodic break that results in excitation. A ramp period (e.g., the time from beginning of the ramp to the end of the ramp) may be up to 100 milliseconds or even greater. In some examples, a ramp period of about 10 millisecond (or greater) may be enough for blocking A neurons. In some examples, a ramp period of greater than about 100 milliseconds for the ramp period may be appropriate for blocking C neurons. Example ramp periods include first period (duration) 66 for the ramp up of cathodic phase 52 of pulse 50 and second period 68 for the ramp down of cathodic phase 52 in FIG. 4A. Although not labelled in FIG. 4A, anodic phase 54 of pulse 50 also includes a ramp period for the ramp up and another ramp period for the ramp down. The ramp may be linear (e.g., as shown for the four ramp periods in FIG. 4A for pulse 50), curvilinear, or stepped in profile. For each of cathodic phase 52 and anodic phase 54, the ramp period and/or rate of amplitude change may be the same or different for the ramp up and ramp down portions. The ramp down and ramp up for cathodic phase 52 may be the same or different compared to the ramp down and ramp up for anodic phase 54.

In some examples, for each of cathodic phase 52 and anodic phase 54, the length (duration 62 for cathodic phase 52 and duration 64 for anodic phase 54) may be up to about 10 seconds or even greater. The length/duration of each phase 52, 54 may be short enough to prevent chemical reaction that may be deleterious to the electrode material.

FIG. 4B is a timing diagram showing electrical stimulation 70 that includes a plurality of bi-phasic pulses 50A-50D delivered in a continuous and repeating fashion. Respective bi-phasic pulses 50A-50D may each be an example of pulse 50 described herein and shown in FIG. 4A. As noted above, pulses 50A-50D may be delivered at a relatively low frequency (e.g., about 4 Hz or less, such as 2 Hz or 1 Hz or lower).

As show in FIG. 4B, in some examples, there is substantially no delay between respective bi-phasic pulses 50A-50D of the ULF stimulation. For example, at time t(2), there is substantially no delay between the end of pulse 50A and the beginning of pulse 50B. In other examples, there is a delay between one or more of respective pulses 50A-5D, e.g., during which time carry over effects from the ULF stimulation may still provide a block to the neural activity. For example, there may be a delay of at least about 1 millisecond (msec) or at least about 2 msec, such as at least about 5 msec, between the end of a pulse such as pulse 50A and the beginning of the next respective pulse such as pulse 50B where the amplitude is zero or approximately zero. In some examples, the carry-over effects increase with time that stim is applied, e.g., with less initially but more at later times, so that the delay may start at about 1 to about 2 msec and then be increased to about 5 milliseconds. Hence, there may an immediate transition (i.e., no interval) between the anodic phase 54A of pulse 50A and the cathodic phase 52B of pulse 50B at t(2), or there may be an interval following the anodic phase 54A of pulse 50A and the cathodic phase of pulse 50B. Likewise, an interphase interval may or may not be present between the respective phases of the bi-phasic pulses 50A-50D (e.g., between cathodic phase 52A and anodic phase 54A of pulse 50A in FIG. 4B). In some examples where there is an interphase interval for one or more of pulses 50A-50D, the interval (time duration) may be at least about 0.5 msec such as about 2 sec to about 30 minutes.

Any suitable technique may be used to define the various parameters for the waveform of pulse 50. For example, when defining phase parameters of bi-phasic pulse 50 of FIG. 4A, one may start with cathodic phase 52 and find a limit of amplitude 60 and length 62 (i.e., duration or width) that does not damage the electrode by way of induced chemical reaction and then define anodic phase parameters (e.g., amplitude 58 and length 64) based on cathodic phase 52 (e.g., such that the anodic phase 54 and cathodic phase 52 are substantially charged balanced) and at an amplitude 58 that is effective in blocking the neural activity. In some embodiments, a slight misbalance in stimulation may be beneficial in maintaining electrochemical balance of the electrode. For example, if the average voltage of the electrode has shifted relative to the reference electrode, temporary application of misbalanced stimulation (e.g. 1 microampere (μA), 10 μA, or 100 μA) in the opposite direction to the misbalance may bring the electrode back to neutral electrochemical state. In other example, the respective phases have the same charges so that phases 54 and 52 are charged balanced.

In some examples, bi-phasic stimulation such as that shown in FIG. 4B may have a frequency (e.g., the pulse rate at which pulses 50A-50D are delivered over a period of time) and amplitude for each respective phase of pulses 50A-50D (e.g., amplitudes 58 and 60 in FIG. 4A) that is selected such that the delivery of the stimulation blocks activity from traveling along nerve fibers influenced by the electrical stimulation (e.g., by blocking activity from traveling along A and/or C fibers). For example, 500 μA may be necessary to block activity during the cathodic phases of the waveform 52, while 800 μA may be necessary to block activity during anodic phases of the waveform 54. In general, in some examples, block may be achieved with amplitudes from 100 μA to 10 mA for either cathodic or anodic phases.

In some examples, Waveform 1 for pulse 50 in FIG. 4A (as well as the other waveforms described herein) may be delivered by IMD 14 via an electrode combination comprising at least two electrodes (one or more operating as an anode and one or more operating as a cathode, e.g., on an alternating basis for delivery of bi-phasic stimulation).

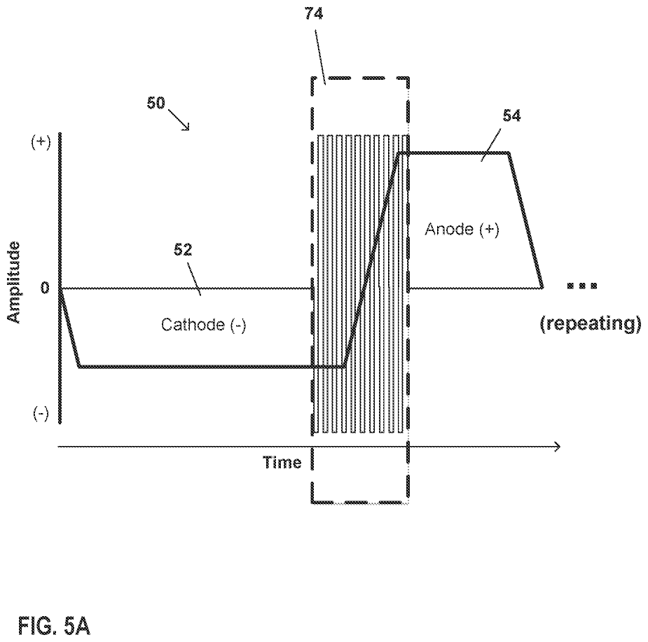

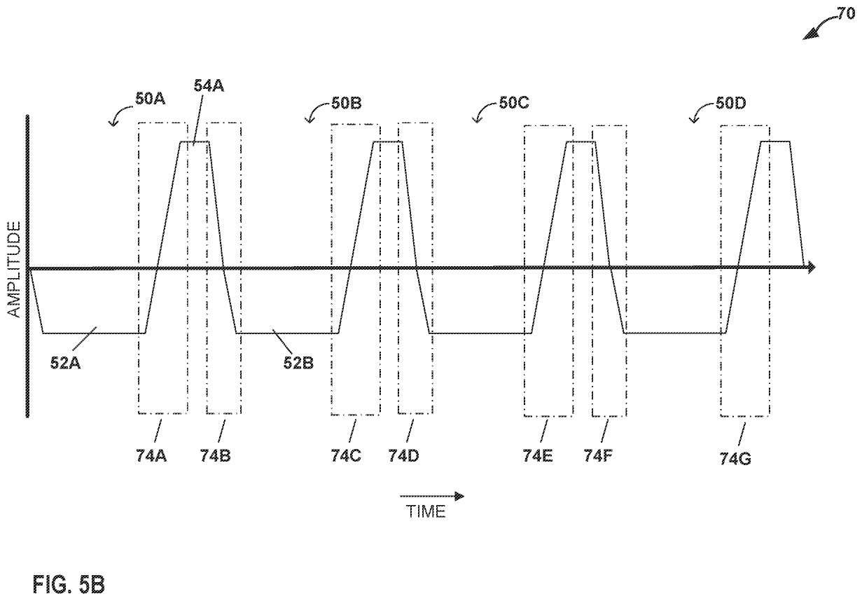

FIG. 5A is another example timing diagram showing the delivery of a single ultra-low frequency pulse 50 (bi-phasic pulse) in combination with the delivery of higher frequency (HF) pulses (not individually labelled) during the time period 74 shown in FIG. 5 so that time period 74 of the HF stimulation is overlapping or otherwise delivered in combination with each single ultra-low frequency pulse 50, in accordance with examples of the disclosure. For example, in FIG. 5A, time period 74 during which the HF pulses are delivered begins during cathodic phase 52 and ends during anodic phase 54. However, in this example, time period 74 does not extend over the entirety of pulse 50. Such overlap may be necessary to avoid onset response of fibers associated with HF pulses. In some examples, the onset of block due to the HF stimulation is fairly fast, e.g., after first spike/pulse of the stimulation. In such examples, the overlap short of the HF stimulation 74A with, e.g., the end of cathodic phase 52A may be about 10 millisecond or less, such as 5 milliseconds or less. In some example, the HF stimulation during period 74A may not start until after the ramp down of cathodic phase 52A.

Although the individual pulses are not labelled in FIG. 5A, during the higher frequency time period 74 shown in FIG. 5, a higher frequency train of pulses may be delivered at a higher frequency (e.g., at least 1 kHz, such as about 1 kHz to about 50 kHz, or about 5 kHz to about 10 kHz) with alternating polarity and with the train of pulses being substantially charge balanced. Examples of the disclosure may include the continuous delivery of such HF pulses in a repeating fashion (e.g., with or without a time delay between the end of the ultra-low frequency (ULF) pulse 50 shown in FIG. 5A and the next subsequent ULF pulse having the same waveform). The bi-phasic ULF pulse 50 shown in FIG. 5 may be the same as or similar to pulse 50 shown in FIG. 4A. The waveform shown in FIG. 5 may be referred to as “Waveform 2.”

For Waveform 2, the HF pulses delivered during time period 74 may be used to produce a block (e.g., block of nerve propagation or activation) to ensure that neurons are continued to be blocked in transition from the cathodic phase 52 to anodic phases 54 of the ULF pulse 50. The same or similar HF stimulation may be delivered at the transition between the anodic phase 54 of the ULF pulse 50 shown in FIG. 5A and between the cathodic phase of the next bi-phasic (ULF) pulse being delivered (not shown). Such a transition would include time t(2) at the transition between anodic phase 54A of pulse 50A and the cathodic phase (not labelled) of pulse 50B in the timing diagram of FIG. 4B. In these transitions, the IMD may apply a high-rate stimulus to block neurons.

FIG. 5B is a timing diagram showing the delivery of ULF pulses 50A-50D in combination with high rate/HF pulses during time periods 74A-74G. ULF pulses 50A-50D may be the same or substantially similar to that described above with regard to FIG. 4B. As shown, respective periods of time periods 74A-74G overlap with either the transition between cathodic and anodic phases of each respective pulse 50A-50D (such as time period 74A overlapping with the end of cathodic phase 52A and the beginning of anodic phase 54A of pulse 50A) or the transition between the end and beginning of neighboring pulses 50A-50D (such as time period 74B which overlaps with the end of anodic phase 54A of pulse 50A and the beginning of cathodic phase 52B of pulse 50B. The frequency of the pulses delivered during each of time periods 74A-74G may be the same or different.

Thus, Waveform 2 and/or the stimulation shown in FIGS. 5A and 5B may blend in ULF pulses delivered substantially continuously with periodically delivered high rate/higher frequency pulses (e.g., during time period 74 in FIG. 5B and time periods 74A-74G in FIG. 5B). The higher frequency stimulation may be stopped (e.g., periodically as shown between each of time periods 74A-74G) when the blocking takes effect from the other phase of the low frequency stimulation pulse. For example, time period 74A may end when the blocking from anodic phase 54A takes effect. Likewise, time period 74B may end with the blocking from cathodic phase 52B take effect. In some examples, the delivery of the high rate/higher frequency pulses may be terminated based on sensing of nerves signals showing the block has been reestablished or for only a predetermined period of time after which there is a transition back to the delivery of ULF pulses/waveforms only.

In some examples, the high rate/HF stimulation may be adaptively delivered if activity on target nerve fibers is sensed, e.g., by triggering the delivery of the HF pulses based on the sensing of nerve activity during the delivery of the ultra-low frequency pulses 50A-50D on a continuous basis. Sensing of such nerve activity may be performed, for example, by sensing module 32 of IMD 14. In an example, processor 26 of IMD 14 may control delivery of the stimulation, such as the delivery of the HF pulses to overlap or coincide with one or more of pulses 50A-50D, based on sensing of nerve activity (e.g., to determine whether a desired nerve block is occurring) during the delivery of the ULF pulses by sensing module 32. The HF stimulation (e.g., the stimulation delivered during time period 74 in FIG. 5A and time periods 74A-74G in FIG. 5B) may have a frequency and amplitude such that the stimulation blocks activity from traveling along A and/or C fibers. In some examples, the individual pulses of the HF stimulation may have a pulse width/duration of approximately 200 microseconds or greater, and less than half the overall period of the HF stimulation. The amplitude (e.g., current amplitude) of the HF stimulation may be greater than the amplitude of the ULF stimulation, e.g., at least about 50% greater than the amplitude of the ULF stimulation or at least about two times the amplitude of the ULF stimulation.

If delivered alone without ULF pulses, the delivery of HF stimulation such as that delivered during time period 74 or 74A-74G may cause onset of neural activity when initiated. However, in Waveform 2 of FIG. 5A or the electrical stimulation shown in FIG. 5B, the ULF stimulation pulse(s) 50 or 50A-50D may already be blocking the neural activity so that there is no onset response to the HF stimulation during periods 74 and 74A-74G. In some examples, the HF stimulation is only used during the transition region between the alternating phases of ULF stimulation pulses such as that shown in FIGS. 5A and 5B. The HF stimulation may start while there is still a block by the ULF stimulation, e.g., where the HF stimulation starts before ramp down or during ramp down of the cathodic and/or anodic phase, and the HF stimulation ends after ramp up or during ramp up and block caused by the ULF stimulation pulse is back. In some examples, the ULF simulation and HF stimulation may be delivered from IMD 14 by the same or different electrode combination (e.g., same or different electrode vector). In some examples, the HF stimulation during time periods 74 and 74A-74G includes pulses delivered at a pulse frequency of about 1 kHz to about 50 kHz, such as about 5 to about 10 kHz. The amplitude of the HF pulses may be greater than the amplitude of the ULF pulses.

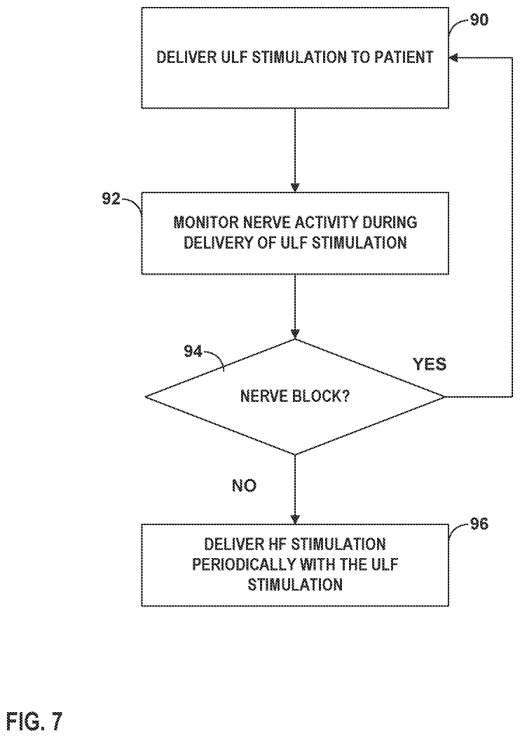

FIG. 7 is a flow diagram illustrating an example technique for delivering electrical stimulation to patient that includes ULF stimulation pulses in combination with HF pulses (e.g., at a frequency of at least 1 kHz), where the HF pulses are delivered in response to sensed nerve activity of the patient during the delivery of the ULF stimulation pulses. For ease of description, the technique of FIG. 7 will be described with regard to system 10 of FIG. 1 and the electrical stimulation represented by the timing diagrams of FIGS. 4B and 5B. However, the example technique of FIG. 7 may be utilized by any suitable medical device system configured to deliver electrical stimulation to a patient.

As indicated in FIG. 7, IMD 14 may deliver ULF electrical stimulation to patient 12 via one or more electrodes located on lead 16, where the ULF electrical stimulation includes a plurality of bi-phasic pulses delivered at a relatively low frequency (90). For example, IMD 14, under the control of processor 26 and using electrode(s) on lead 16, may deliver ULF electrical stimulation including pulses 50A-50D in the manner shown in FIG. 4B on a substantially continuous basis to a target site of patient 12. The target nerve site may be a peripheral nerve site. As described herein, in some examples, the ULF stimulation may be configured to block nerve activity of patient 12 at or near the target site. For example, pulses 50A-50D may block neural activity, (e.g., electrical neuropotentials), from being conducted along nerve fibers (e.g., from one end of the fiber to the other). The block may include one or more of A, B, or C fibers.

During the substantially continuous delivery of the ULF electrical stimulation, IMD 14, using sensing module 32 and one or more electrodes on lead 16, may monitor the nerve activity of patient 12, e.g., at or near the target site, to determine if the ULF electrical stimulation successfully blocks the nerve activity of patient 12. IMD 14 may monitor the nerve activity using any suitable technique including techniques for sensing electrical nerve activity of patient 12.

If processor 26 of IMD 14 determines that the continuous delivery of the ULF stimulation including pulses 50A-50D in FIG. 4B successfully blocks the targeted nerve activity, e.g., based on the periodic sensing of nerve activity occurring during the transition between respective pulses and/or respective phases within a pulse, then IMD 14 may continue to deliver the ULF stimulation pulses without adding the delivery of HF stimulation pulses (90). Conversely, if IMD 14 determines that the continuous delivery of the ULF stimulation including pulses 50A-50D in FIG. 4B is not successfully blocking the targeted nerve activity (e.g. based on the periodic sensing of nerve activity occurring during the transition between respective pulses and/or respective phases within a pulse), then IMD 14 may initiate the delivery of HF stimulation pulses in combination with the delivery of the ULF stimulation pulses (96). For example, IMD 14, under the control of processor 26, may begin to deliver HF stimulation pulses on a periodic basis with the ULF stimulation pulses, e.g., as shown in FIG. 5B with ULF pulses 50A-50D and HF stimulation pulses during periods 74A-74G.

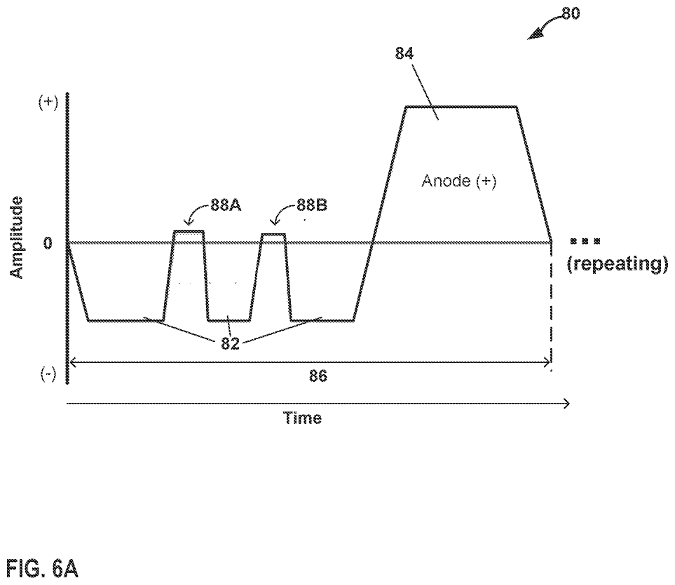

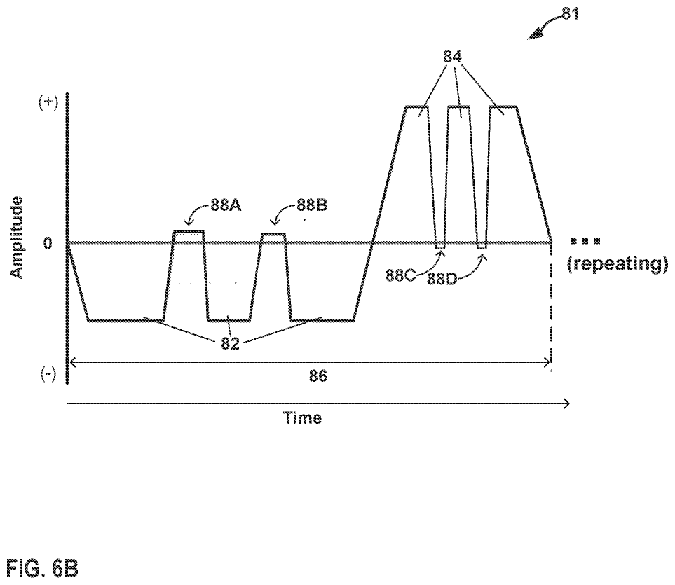

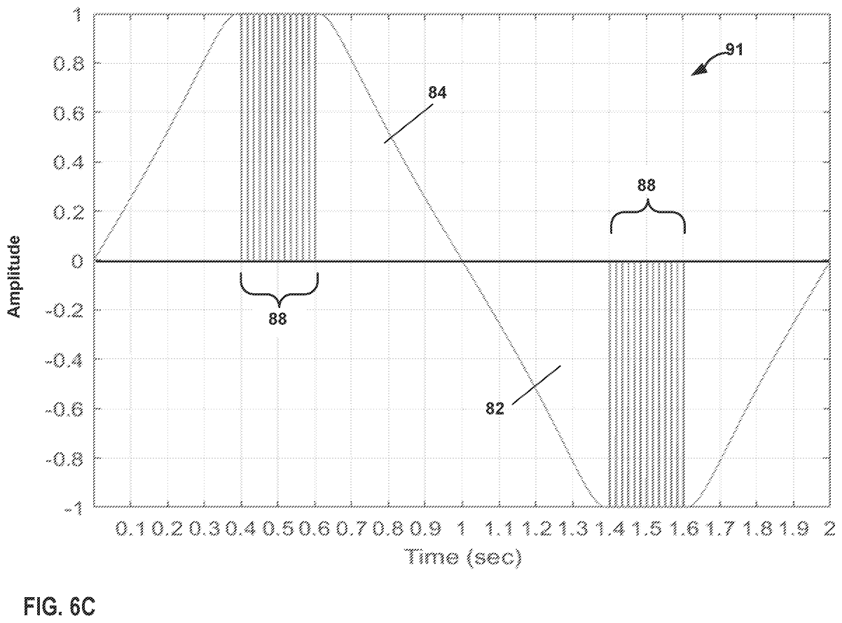

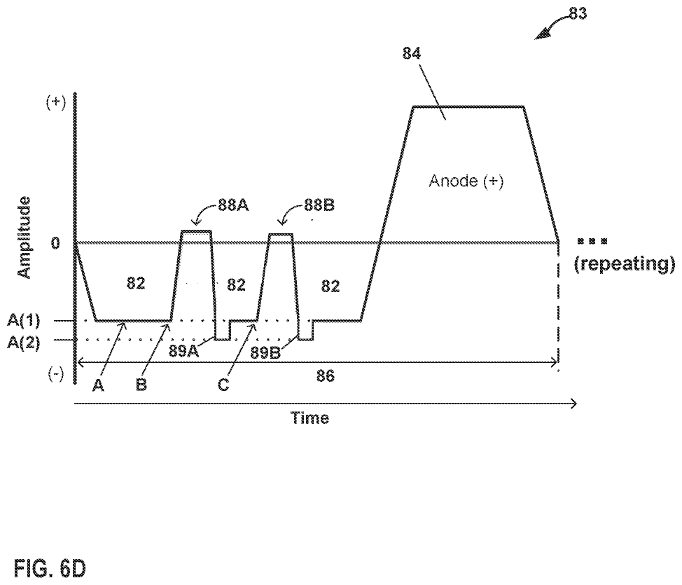

FIG. 6A is an example timing diagram showing the delivery of another example, single ultra-low frequency pulse 80 (bi-phasic pulse) in accordance with examples of the disclosure. Pulse 80 includes cathodic phase 82 and anodic phase 84. Examples of the disclosure may include the continuous delivery of pulse 80 in a repeating fashion (e.g., without or without a time delay between the end of pulse 80 shown in FIG. 6A and the next subsequent pulse having the same waveform). In some examples, pulse 80 may be delivered in place of pulses 50A-50D in the electrical stimulation represented by the timing diagrams of FIGS. 4A and 5A.

In some examples, since the bi-phasic pulse 80 has a relatively long pulse width or duration (e.g., where duration 86 is at least about 0.25 seconds), the repeated and continuous delivery of bi-phasic pulse 50 or 80 may be at a relatively low frequency. The example waveform in FIG. 6A may be referred to as “Waveform 3.”

Waveform 3 for pulse 80 may be substantially similar to Waveform 1 for pulse 50 but with one or more “gaps” in one or both of the phases 82 and 84. In FIG. 6A, cathodic phase 82 is shown with two “gaps” 88A and 88B in which the amplitude is temporarily reduced to zero or even set to an amplitude level of the opposite polarity (e.g., with each gap 88A and 88B having a period of time in which the amplitude is zero and/or a period of time in which there is a small amplitude, anodic polarity portion).

During each gap 88A and 88B, there may be at least a partial release of the block of the nerve fibers otherwise blocked by the delivery of the stimulation during the cathodic phase 82. Each gap 88A and 88B may be configured to break a block caused by the stimulation of cathodic phase 82 and allow conduction for fast fibers (e.g., A fibers) but not slow (e.g., C fibers) fibers during gap(s) 88A and 88B. For example, each gap 88A and 88B may be short enough in duration to break a block caused by the stimulation of cathodic phase 82 and allow conduction for fast fibers (e.g., A fibers) but not slow (e.g., C fibers) fibers during gap(s) 88A and 88B. A-fibers may recover relatively fast during gaps 88A and 88B so that if there is a neural signal coming during gap 88A or 88B, the signal will pass rather than being blocked. Conversely, slower fibers such as C-fibers may recover from the block slower and continue to be blocked during gaps 88A and 88B.

Such gaps can be repeated at regular or irregular intervals during the continuous and repeated delivery of pulse 80 (e.g., in the manner shown in FIG. 4B for pulse 50) to allow neural information to pass through. During gap 88A and/or 88B, to avoid anode break excitation, there may be an onset ramp (e.g., the ramp at the beginning of each of gap 88A and 88B waveform) (especially for A fibers) and/or an offset ramp (e.g., the ramp at the end of each gap 88A and 88B waveform). In some examples, the length of each individual gap 88A and 88B in the bi-phasic pulse may be on the order of up to about 2 milliseconds, e.g., greater than 0 but less than or equal to about 2 milliseconds. The length of the gap may be selected to take advantage of the slow response rate of C fibers compared to A fibers, but to not give the C fibers enough time to reset to baseline. While pulse 80 (and other waveforms with such gaps occurring in the one or more other the phases) may be referred to as an ultra-low frequency pulse, the waveform may not necessarily be ULF because the cathodic phase 82 is no longer in a single polarity throughout the entire duration of the phase.

In some examples, gaps 88A and 88B may be in the cathodic phase 82 or anodic phase 84, or both, of the ULF bi-phasic pulse 80. FIG. 6A illustrates an example timing diagram showing two gaps in cathodic phase 82 without one or more gaps in anodic phase 84. Conversely, FIG. 6B illustrates an example timing diagram like the timing diagram of FIG. 6A but with two gaps in both the cathodic phase 82 and the anodic phase 84 of pulse 81. While two separate gaps are shown for the cathodic phase 82 in FIG. 6A and each of the cathodic phase 82 and the anodic phase 84, there may be fewer gaps (one) or more (more than two) within a phase in other examples. In some examples, the respective gaps (such as gaps 88A and 88B) may occur up to every 3 milliseconds, such as, up to every 10 milliseconds, to allow neural flow to recover. The gaps may occur at a regular or irregular frequency within a phase.

For the example “gaps” (such as gaps 88A and 88B), the stimulation amplitude may go to zero or reverse polarity, e.g., slightly, to release a block for A fibers but not C fibers. This may allow conduction of sensory information to the patient during the gaps. The amplitude may be ramped back up again when C fibers become unblocked or just before the C fibers become unblocked. The reverse polarity may be provided to help prevent damage to the electrode due to chemical reaction (e.g., and possibly allow for longer cathodic and/or anodic phase).

FIG. 6C illustrates another example timing diagram like the timing diagram of FIG. 6B with gaps 88 in both the cathodic phase 82 and the anodic phase 84 of pulse waveform 91. However, in the timing diagram of FIG. 6C, anodic phase 84 is delivered prior to cathodic phase 82 and the amplitude and length of each phase is approximately equal. Furthermore, anodic phase 84 and cathodic phase 82 each includes a plurality of gaps 88 (not individually labelled) during which the amplitude temporarily is zero (e.g., rather than a nominal amplitude of the opposite polarity). The number of individual gaps 88 that occur during each phase 82 and 84 is more than two. For example, the timing diagram of FIG. 6C shows each respective phase 82 and 84 being approximately 1 second in length and thirteen individual gaps 88 occurring in the middle of each respective phase at a frequency of about 65 Hertz (Hz). In some examples, plurality of gaps 88 may occur at a frequency of at least about 2, Hz or at least about 20 Hz, such as at least about 50 Hz, about least about 75 Hz, at least about 100 Hz, or about 2 Hz to about 1000 Hz. The frequency of the gaps 88 may be selected to allow for unblock of fast fiber activity such as A-fiber activity, as described herein. Other values that those described are contemplated. The total number of individual gaps may depend on the frequency of the respective gaps as well as the length of the gaps and overall length of a respective phase during which the gaps occur.

FIG. 6D is an example timing diagram showing the delivery of another example, single ultra-low frequency pulse 83 (bi-phasic pulse) waveform in accordance with examples of the disclosure. Pulse 83 may be substantially similar to pulse 80 of FIG. 6A and includes cathodic phase 82 and anodic phase 84, with cathodic phase 82 including gaps 88A and 88B. In the example of FIG. 6D, during gaps 88A and 88B, the stimulation amplitude is shown in reverse polarity slightly.

However, unlike pulse 80 in FIG. 6A, pulse 83 includes overshoot portion 89A following the end of gap 88A and overshoot portion 89B following the end of gap 88B. For each of overshoot portions 89A and 89B, the stimulation amplitude is temporarily increased above the amplitude of cathodic phase 82. The amplitude of the cathodic phase may be defined by the amplitude of cathodic phase 82 prior to the first occurrence of a gap (e.g., the amplitude at of the plateau including point A prior to gap 88A shown in FIG. 6D) and/or the amplitude of the cathodic phase 82 just prior to the onset of the gap which is ending with the overshoot portion (e.g., the amplitude at point B just prior to the occurrence of gap 88A and the amplitude at point C just prior to the occurrence of gap 88B).

In the example of FIG. 6D, the amplitude at each of points A, B, and C is amplitude A(1) and the amplitude of each of overshoot portions 89A and 89B is amplitude A(2), although the amplitude of overshoot portion 89A may be different than overshoot portion 89B in some instances. In some examples, the difference between amplitude A(1) and amplitude A(2) may be at least about 1% of A(1), or about 0.1% to about 10% of the amplitude of A(1), or where A(2) is about 1% greater than A(1), or about 0.1% to about 10% greater than A(1).

The increased amplitude during overshoot portions 89A and 89B may be included to prevent a response to the stimulation in which slower nerve fibers, such as, C-fibers are temporarily unblocked. For example, even though the gaps 88 occur in a manner in which the slower fibers (C-fibers) do not respond (e.g., are not unblocked) for each individual gap, on average during the delivery of a plurality of pulses (e.g., as shown in FIGS. 4B and 5B), the slower fibers may be undesirably unblocked in addition to the unblock of faster fibers (A-fibers) during the occurrence of one or more individual gaps of the plurality of gaps 88. The amount of “unblock” may be dependent on rate/frequency of gaps 88, duration of gaps 88, and/or the shape/morphology of gaps 88.

To prevent such a possibility, an amplitude overshoot may be present in one or more phases of one or more pulses of a stimulation therapy. In the example of FIG. 6D, the amplitude overshoot is included on a gap by gap basis for one or more of gaps 88A or 88B by including overshoot portions 89A and 89B, respectively. Amplitude overshoot 89A may prevent undesired unblock of slower fibers during the next gap 88B or may reinforce or reestablish the block of the slower fibers that may have lessened or ceased during gap 88A. An amplitude overshoot such as overshoot 89A and 89B may be present for all or only some of the gaps occurring in an anodic phase 84 and/or cathodic phase 82 of a pulse. In a series of pulses delivered, e.g., as shown in FIGS. 4B and 5B), an amplitude overshoot may be present in some but not all of the pulses and may be present in one or both of the cathodic and anodic phases of the pulses including an amplitude overshoot. Overshoot 89A may extend for only a portion of time between the end of gap 89A and the beginning of gap 89B, or may extend for substantially the entire time between the end of gap 89A and the beginning of gap 89B.

In addition, rapid onset of blocking during the gap, may induce spikes in fast A fibers, which may not be sufficiently blocked by the cathodic plateau. To avoid this possibility, the amplitude overshoot may be necessary to rapidly re-establish block and avoid propagation of generated spiking activity in A fibers due to the gaps.

In some examples, for a series of pulses, an amplitude overshoot that is included for the gaps may in one or both of the phases may be slightly increased over time, e.g., with the magnitude of the amplitude of the overshoot of the first pulses or earlier pulses in a series being lower than the magnitude of the amplitude overshoot for the second or later pulses in a series of pulses. For example, in a series of pulses like that shown in FIG. 4B, one or both phases 52A and 54A of pulse 50A may include one or more gaps without an amplitude overshoot or with an overshoot having a magnitude of X₁, one or both phases of pulse 50B may include one or more gaps with an overshoot having a magnitude of X₂ that is greater than X₁, one or both phases of pulse 50C may include one or more gaps with an overshoot having a magnitude of X₃ that is greater than X₂, and so forth. In some examples, within a single phase of a pulse, the magnitude of the amplitude overshoot may be increased over time, e.g., with the magnitude of overshoot portion 89A being less than the magnitude of overshoot portion 89B in FIG. 6D.

Thus, to compensate for the undesired unblocking of slower fiber activity from the occurrence of gaps 88, e.g., the cumulative occurrence of gaps 88, an overshoot in the amplitude may be applied on a pulse by pulse basis. Additionally, or alternatively, a global adjustment to the amplitude of one or both phases of each pulse in a series of pulses may be made by increasing the overall amplitude of the one or both phases account for the possible undesirable unblocking of slower fiber activity resulting from gaps 88.

Although not shown in FIGS. 6A-6D, the periodic HF stimulation of FIGS. 5A and 5B may be applied during the stimulation represented in FIGS. 6A-6D.

Examples of the disclosure may include delivering stimulation therapy to one or more locations to treat sensory or motor disorders characterized by overactive nerve activity, but the treatment of other types of disorders is contemplated. For example, examples of the described stimulation may be delivered as therapies to treat one or more other patient conditions, such as, e.g., voiding disorders, bowel movement disorders, spastic colon, irritable bowel syndrome (IBS), interstitial cystitis, autonomic disorders, (such as, hypertension, hyperhidrosis), epilepsy, Parkinson's disease, Alzheimer's disease, dystonia, schizophrenia, obsessive compulsive disorder, and depression. Accordingly, in some examples, the stimulation may be delivered to neural tissue in the brain, spinal cord, digestive system, or pelvic region.

In some examples, the stimulation may be used to block pathological nerve activity (e.g., to treat pain disorders) or block sensory activity (e.g., to treat sensory disorders).

Furthermore, in some examples, the described higher frequency (HF) and/or ultra-low frequency (ULF) stimulation may be delivered to more than one nerve site or different sites. For example, in the case of ULF stimulation being delivered at a location different from that of the HF stimulation, the HF stimulation and/or ULF stimulation may be delivered to multiple nerve sites along connected neural pathways. In some examples, HF stimulation may be delivered to multiple branches of a nerve in combination with the delivery of ULF stimulation to the trunk of the nerve, or vice versa. For example, for pudendal nerve stimulation, ULF stimulation may be delivered to the pudendal nerve trunk and HF stimulation may be delivered to nerve sites on two or more pudendal branches, e.g., dorsal genital nerve, perineal nerve, inferior rectal nerve. The HF stimulation could be delivered to each branch at the same time or individually, e.g., based on pain being experienced by a patient.

The techniques described in this disclosure may be implemented, at least in part, in hardware, software, firmware or any combination thereof. For example, various aspects of the techniques may be implemented within one or more microprocessors, DSPs, ASICs, FPGAs, or any other equivalent integrated or discrete logic circuitry, as well as any combinations of such components, embodied in programmers, such as physician or patient programmers, stimulators, or other devices. The term “processor” or “processing circuitry” may generally refer to any of the foregoing logic circuitry, alone or in combination with other logic circuitry, or any other equivalent circuitry.

When implemented in software, the functionality ascribed to the systems and devices described in this disclosure may be embodied as instructions on a computer-readable medium such as RAM, ROM, NVRAM, EEPROM, FLASH memory, magnetic media, optical media, or the like. The instructions may be executed to support one or more aspects of the functionality described in this disclosure.

If implemented in software, the techniques described in this disclosure may be stored on or transmitted over as one or more instructions or code on a computer-readable medium. Computer-readable media may include non-transitory computer storage media or communication media including any medium that facilitates transfer of a computer program from one place to another. Data storage media may be any available media that can be accessed by one or more computers or one or more processors to retrieve instructions, code and/or data structures for implementation of the techniques described in this disclosure. By way of example, and not limitation, such data storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

The code may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

In addition, it should be noted that the systems described herein may not be limited to treatment of a human patient. In alternative examples, these systems may be implemented in non-human patients, e.g., primates, canines, equines, pigs, birds, and felines. These animals may undergo clinical or research therapies that my benefit from the subject matter of this disclosure.

Various examples of the disclosure have been described. Various modifications may be made without departing from the scope of the claims. These and other examples are within the scope of the following numbered examples and claims.

Example 1. A method comprising controlling, using processing circuitry, the delivery of an electrical stimulation therapy to a patient via a medical device, wherein the electrical stimulation therapy includes a plurality of bi-phasic pulses, each pulse of the bi-phasic pulses including a first phase followed by a second phase, and wherein the plurality of bi-phasic pulses are configured to substantially block transmission of neural activity along nerve fibers of the patient.

Example 2. The method of example 1, wherein controlling the delivery of the electrical stimulation therapy includes controlling the delivery of the electrical stimulation such that the delivered electrical stimulation therapy includes one or more gaps during at least one of the first phase or the second phase of respective bi-phasic pulses, and wherein an amplitude during the one or more gaps is about zero or a reverse polarity of the amplitude of at the respective first or second phase.

Example 3. The method of example 2, wherein the one or more gaps are configured to temporarily release a block of A-fibers caused by the plurality of bi-phasic pulses.

Example 4. The method of example 2 or 3, wherein the one or more gaps are configured to not release a block of C-fibers caused by the plurality of bi-phasic pulses.

Example 5. The method of any one of examples 2-4, wherein the delivered electrical stimulation therapy includes an amplitude overshoot following the one or more gaps occurring during the at least one of the first phase or the second phase of respective bi-phasic pulses.

Example 6. The method of any one of examples 2-5, wherein controlling the delivery of the electrical stimulation such that the delivered electrical stimulation therapy includes the one or more gaps during the at least one of the first phase or the second phase of the respective bi-phasic pulses comprises controlling the delivery of the electrical stimulation such that the delivered electrical stimulation therapy includes the one or more gaps during both of the first phase or the second phase.

Example 7. The method of any one of examples 2-6, wherein controlling the delivery of the electrical stimulation such that the delivered electrical stimulation therapy includes the one or more gaps during the at least one of the first phase or the second phase of the respective bi-phasic pulses comprises controlling the delivery of the electrical stimulation such that an amplitude of the delivered electrical stimulation is ramped down at an onset of a respective gap of the one or more gaps and ramped up at an end of the respective gap of the one or more gaps.

Example 8. The method of any one of examples 1-7, further comprising controlling, using the processing circuitry, delivery of higher frequency stimulation pulses having a frequency higher than the frequency of the bi-phasic pulses in combination with the plurality of bi-phasic pulses, wherein the higher frequency pulses overlap with at least one of a transition between the first phase and the second phase in each respective bi-phasic pulse of the plurality of bi-phasic pulses or a transition between the respective pulses of the plurality of bi-phasic pulses, and wherein the delivery of the higher frequency stimulation pulses is configured to substantially block transmission of neural activity along nerve fibers.

Example 9. The method of example 8, wherein the higher frequency stimulation pulses are delivered with a frequency of at least about 1 kHz.

Example 10. The method of examples 8 or 9, further comprising sensing nerve activity of the patient during the delivery of the plurality of bi-phasic pulses, wherein the delivery of the higher frequency stimulation pulses is initiated in response to the sensing of the nerve activity of the patient.

Example 11. The method of any one of examples 1-10, wherein the plurality of bi-phasic pulses are delivered at a frequency of about 0.01 Hz to about 10 Hz.

Example 12. The method of any one of examples 1-11, wherein the bi-phasic pulses are asymmetric with the first phase having a longer duration and a lower amplitude compared to the second phase.

Example 13. The method of example 12, wherein the first phase is a cathodic phase and the second phase is an anodic phase.

Example 14. The method of any one of examples 1-13, wherein respective pulses of the bi-phasic pulses are substantially charge balanced.

Example 15. The method of any one of examples 1-14, wherein controlling, using the processing circuitry, the delivery of an electrical stimulation therapy to the patient via the medical device includes controlling, during a transition from the first phase to the second phase of each respective pulse, an amplitude of the stimulation by ramping the amplitude over a ramp period.

Example 16. The method of example 15, wherein the ramp period is at least about 10 milliseconds.

Example 17. A medical device system comprising: a therapy module configured to deliver electrical stimulation therapy to a patient; and processing circuitry configured to control the therapy module to deliver the electrical stimulation to the patient such that the electrical stimulation therapy includes a plurality of bi-phasic pulses, each pulse of the bi-phasic pulses including a first phase followed by a second phase, and wherein the plurality of bi-phasic pulses are configured to substantially block transmission of neural activity along nerve fibers of the patient.

Example 18. The system of example 17, wherein the processing circuitry is configured to control the therapy module to delivery of the electrical stimulation therapy such that the delivered electrical stimulation therapy includes one or more gaps during at least one of the first phase or the second phase of respective bi-phasic pulses, and wherein an amplitude during the one or more gaps is about zero or a reverse polarity of the amplitude of at the respective first or second phase.

Example 19. The system of example 18, wherein the one or more gaps are configured to temporarily release a block of A-fibers caused by the plurality of bi-phasic pulses.

Example 20. The system of example 18 or 19, wherein the one or more gaps are configured to not release a block of C-fibers caused by the plurality of bi-phasic pulses.

Example 21. The system of any one of examples 18-20, wherein the processing circuitry is configured to control the therapy module to delivery of the electrical stimulation therapy such that the delivered electrical stimulation therapy includes an amplitude overshoot following the one or more gaps occurring during the at least one of the first phase or the second phase of respective bi-phasic pulses.

Example 22. The system of any one of examples 18-21, wherein the processing circuitry is configured to control the therapy module to delivery of the electrical stimulation therapy such that the delivered electrical stimulation therapy includes the one or more gaps during both of the first phase or the second phase.

Example 23. The system of any one of examples 18-22, wherein the processing circuitry is configured to control the therapy module to delivery of the electrical stimulation therapy such that an amplitude of the delivered electrical stimulation is ramped down at an onset of a respective gap of the one or more gaps and ramped up at an end of the respective gap of the one or more gaps.

Example 24. The system of any one of examples 17-23, wherein the processing circuitry is configured to control the therapy module to deliver higher frequency stimulation pulses having a frequency higher than the frequency of the bi-phasic pulses in combination with the plurality of bi-phasic pulses, wherein the higher frequency pulses overlap with at least one of a transition between the first phase and the second phase in each respective bi-phasic pulse of the plurality of bi-phasic pulses or a transition between the respective pulses of the plurality of bi-phasic pulses, and wherein the delivery of the higher frequency stimulation pulses is configured to substantially block transmission of neural activity along nerve fibers.

Example 25. The system of example 24, wherein the higher frequency stimulation pulses are delivered with a frequency of at least about 1 kHz.

Example 26. The system of examples 24 or 25, further comprising a sensing module configured to sense nerve activity of the patient during the delivery of the plurality of bi-phasic pulses, wherein the processing circuitry is configured to initiate the delivery of the higher frequency stimulation pulses by the therapy module in response to the sensing of the nerve activity of the patient.

Example 27. The system of any one of examples 17-26, wherein the plurality of bi-phasic pulses are delivered at a frequency of about 0.01 Hz to about 10 Hz.

Example 28. The system of any one of examples 17-27, wherein the bi-phasic pulses are asymmetric with the first phase having a longer duration and a lower amplitude compared to the second phase.

Example 29. The system of example 28, wherein the first phase is a cathodic phase and the second phase is an anodic phase.

Example 30. The system of any one of examples 17-29, wherein respective pulses of the bi-phasic pulses are substantially charge balanced.

Example 31. The system of any one of examples 17-30, wherein controlling, using the processing circuitry, the delivery of an electrical stimulation therapy to the patient via the medical device includes controlling, during a transition from the first phase to the second phase of each respective pulse, an amplitude of the stimulation by ramping the amplitude over a ramp period.

Example 32. The system of example 31, wherein the ramp period is at least about 10 milliseconds.

1. A medical device system comprising:
a therapy module configured to deliver electrical stimulation therapy to a patient; and
processing circuitry configured to control the therapy module to deliver the electrical stimulation to the patient such that the electrical stimulation therapy includes a plurality of bi-phasic pulses, each pulse of the bi-phasic pulses including a first phase followed by a second phase, and wherein the plurality of bi-phasic pulses are configured to substantially block transmission of neural activity along nerve fibers of the patient.

2. The system of claim 1, wherein the processing circuitry is configured to control the therapy module to delivery of the electrical stimulation therapy such that the delivered electrical stimulation therapy includes one or more gaps during at least one of the first phase or the second phase of respective bi-phasic pulses, and wherein an amplitude during the one or more gaps is about zero or a reverse polarity of the amplitude of at the respective first or second phase.

3. The system of claim 2, wherein the one or more gaps are configured to temporarily release a block of A-fibers caused by the plurality of bi-phasic pulses.

4. The system of claim 2, wherein the one or more gaps are configured to not release a block of C-fibers caused by the plurality of bi-phasic pulses.

5. The system of claim 2, wherein the processing circuitry is configured to control the therapy module to delivery of the electrical stimulation therapy such that the delivered electrical stimulation therapy includes an amplitude overshoot following the one or more gaps occurring during the at least one of the first phase or the second phase of respective bi-phasic pulses.

6. The system of claim 2, wherein the processing circuitry is configured to control the therapy module to delivery of the electrical stimulation therapy such that the delivered electrical stimulation therapy includes the one or more gaps during both of the first phase or the second phase.

7. The system of claim 2, wherein the processing circuitry is configured to control the therapy module to delivery of the electrical stimulation therapy such that an amplitude of the delivered electrical stimulation is ramped down at an onset of a respective gap of the one or more gaps and ramped up at an end of the respective gap of the one or more gaps.

8. The system of claim 1, wherein the processing circuitry is configured to control the therapy module to deliver higher frequency stimulation pulses having a frequency higher than the frequency of the bi-phasic pulses in combination with the plurality of bi-phasic pulses, wherein the higher frequency pulses overlap with at least one of a transition between the first phase and the second phase in each respective bi-phasic pulse of the plurality of bi-phasic pulses or a transition between the respective pulses of the plurality of bi-phasic pulses, and wherein the delivery of the higher frequency stimulation pulses is configured to substantially block transmission of neural activity along nerve fibers.

9. The system of claim 8, wherein the higher frequency stimulation pulses are delivered with a frequency of at least about 1 kHz.

10. The system of claim 8, further comprising a sensing module configured to sense nerve activity of the patient during the delivery of the plurality of bi-phasic pulses, wherein the processing circuitry is configured to initiate the delivery of the higher frequency stimulation pulses by the therapy module in response to the sensing of the nerve activity of the patient.

11. The system of claim 1, wherein the plurality of bi-phasic pulses are delivered at a frequency of about 0.01 Hz to about 10 Hz.

12. The system of claim 1, wherein the bi-phasic pulses are asymmetric with the first phase having a longer duration and a lower amplitude compared to the second phase.

13. The system of claim 12, wherein the first phase is a cathodic phase and the second phase is an anodic phase.

14. The system of claim 1, wherein respective pulses of the bi-phasic pulses are substantially charge balanced.

15. The system of claim 1, wherein controlling, using the processing circuitry, the delivery of an electrical stimulation therapy to the patient via the medical device includes controlling, during a transition from the first phase to the second phase of each respective pulse, an amplitude of the stimulation by ramping the amplitude over a ramp period.

16. A method comprising controlling, using processing circuitry, the delivery of an electrical stimulation therapy to a patient via a medical device, wherein the electrical stimulation therapy includes a plurality of bi-phasic pulses, each pulse of the bi-phasic pulses including a first phase followed by a second phase, and wherein the plurality of bi-phasic pulses are configured to substantially block transmission of neural activity along nerve fibers of the patient.

17. The method of claim 16, wherein controlling the delivery of the electrical stimulation therapy includes controlling the delivery of the electrical stimulation such that the delivered electrical stimulation therapy includes one or more gaps during at least one of the first phase or the second phase of respective bi-phasic pulses, and wherein an amplitude during the one or more gaps is about zero or a reverse polarity of the amplitude of at the respective first or second phase.

18. The method of claim 17, wherein the one or more gaps are configured to temporarily release a block of A-fibers caused by the plurality of bi-phasic pulses.

19. The method of claim 17, wherein the one or more gaps are configured to not release a block of C-fibers caused by the plurality of bi-phasic pulses.

20. The method of claim 17, wherein the delivered electrical stimulation therapy includes an amplitude overshoot following the one or more gaps occurring during the at least one of the first phase or the second phase of respective bi-phasic pulses.
